(19) United States
(12) Patent Application Publication
(10) Pub. No.: US 2021/0251611 A1
(43) Pub. Date: Aug. 19, 2021

(54) IDENTIFICATION OF RESPIRATORY PHASES IN A MEDICAL PROCEDURE

(71) Applicants: General Electric Company, Schenectady, NY (US); Wisconsin Alumni Research Foundation, Vladison, WI (US)

(72) Inventors: Jhimli Mitra, Niskayuna, NY (US); Sudhanya Chatterjee, Bangalore (IN); Thomas Kwok-Fah Foo, Clifton Park, NY (US); Desmond Teck Beng Yeo, Clifton Park, NY (US); Bryan Patrick Bednarz, Madison, WI (US); Sydney Jupitz, Madison, WI (US)

(21) Appl. No.: 16/795,220

(22) Filed: Feb. 19, 2020

(51) Int. Cl. A61B 8/08; A61B 5/055; A61B 5/08; A61B 8/13; A61B 6/03; A61B 6/00; A61B 5/00; G01R 33/48

(52) U.S. Cl. CPC A61B 8/5261; A61B 5/055; A61B 5/08; A61B 8/0891; A61B 8/13; G01R 33/4814; A61B 6/032; A61B 6/5247; A61B 6/469; A61B 5/7267; A61B 8/5223

(57) ABSTRACT

The present disclosure relates to automatically determining respiratory phases (e.g., end-inspiration/expiration respiratory phases) in real time using ultrasound beamspace data. The respiratory phases may be used subsequently in a therapy or treatment (e.g., image-guided radiation-therapy (IGRT)) for precise dose-delivery. In certain implementations, vessel bifurcation may be tracked and respiration phases determined in real time using the tracked vessel bifurcations to facilitate respiration gating of the treatment or therapy.

### STATEMENT REGARDING FEDERALLY SPONSORED RESEARCH & DEVELOPMENT

This invention was made with Government support under contract number 1R01CA190298 awarded by the National Institute of Health. The Government has certain rights in the invention.

### BACKGROUND

The subject matter disclosed herein relates generally to identification of respiratory events or phases using imaging techniques.

Various medical procedures benefit from knowledge of both internal and external patient motion. By way of example, certain imaging and treatment protocols are directed to specific, localized regions of interest. In such instances, motion attributable to a beating heart (i.e., cardiac motion) or respiration may impact the procedure.

By way of example, radiation therapy is one such treatment that may be impacted by patient motion that may be attributed to respiration. For instance, radiation therapy typically involves directing a stream of radiation toward an internal region of a patient in which a tumor or similar structure has been identified so as to reduce the size of the tumor. Tracking tumor targets before and during radiation therapy is usually performed using manual or semi-automatic methods that limit the application of such treatment therapies that need to be performed in real-time. In particular, because the region undergoing treatment is internal to the patient, it may be difficult to assess the internal position and/or motion of the tumor in a real-time context, which may limit the ability of the directed radiation to be localized to the tumor.

For instance, conventional radiation therapy implementations may employ scan-converted (i.e., rectilinear coordinates) ultrasound data for image analysis. However, there is a processing time overhead to obtain the scan-converted data from the beam-space data. This processing time overhead is not desirable in the context of a real time system, such as a treatment system where accurate application of the treatment is dependent on real-time accurate knowledge of the position of the anatomic region of interest within a patient.

Further, tracking a target in image guide radiation therapy is usually performed using iterative methods. Such iterative methods, however, impose certain burdens and issues. For example, one challenge associated with iterative methods is the need for good initialization. Further, the choice of optimization method and its parameters pose challenges for generalizability of the method. Lastly, iterative methods tend to have high inference time, again making real time implementations difficult if not infeasible.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment a method is provided for generating a patient-specific respiration model. In accordance with this embodiment, magnetic resonance (MR) image data and ultrasound beamspace data of a patient are acquired over time. In the ultrasound beamspace data, one or more vascular vessel bifurcations are tracked over time. Based on a measure of the tracked vascular vessel bifurcations, one or more respiration phases of the patient are determined over time. A 3D MR volume is generated from the MR image data. For a respective respiration phase of the one or more respiration phases, a 3D MR respiration phase volume is determined for the patient from the 3D MR volume and specific to the respective respiration phase.

In a further embodiment, a method for respiration gating a patient treatment is provided. In accordance with this embodiment, during a treatment procedure, ultrasound beamspace data of a patient is acquired over time. In the ultrasound beamspace data, one or more vascular vessel bifurcations are tracked over time. Based on a measure of the tracked vascular vessel bifurcations, a series of activation respiration phases of the patient are determined during the treatment. One or both of a location or orientation of a target anatomic region are determined during the activation respiration phases of the patient using a 3D MR respiration phase volume for the patient generated prior to the treatment procedure and specific to the activation respiration phase. The treatment is applied to the target anatomic region during the series of activation respiration phases.

In an additional embodiment, an image guided treatment system is provided. In accordance with this embodiment, the image guide treatment system comprises: a memory encoding processor-executable routines and a processing component configured to access the memory and execute the processor-executable routines. The routines, when executed by the processing component, cause the processing component to perform actions comprising: acquiring magnetic resonance (MR) image data and three-dimensional (3D) ultrasound beamspace data of a patient over time; in the 3D ultrasound beamspace data, tracking one or more vascular vessel bifurcations over time; based on a measure of the tracked vascular vessel bifurcations, determining one or more respiration phases of the patient over time; generating a 3D MR volume from the MR image data; for a respective respiration phase of the one or more respiration phases, determining a 3D MR respiration phase volume for the patient from the 3D MR volume and specific to the respective respiration phase; and respiration gating application of a treatment to the patient using the 3D MR respiration phase volume and an indication of respiration phase of the patient determined using 3D ultrasound beamspace data acquired during the application of the treatment.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts a radiation treatment system suitable for image-based guidance, in accordance with aspects of the present disclosure;

FIG. 2 illustrates a magnetic resonance imaging (MM) system, in accordance with aspects of the present disclosure;

FIG. 3 is a block diagram of an ultrasound system, in accordance with aspects of the present disclosure;

FIG. 4 depicts a schematic diagram of an embodiment of a magnetic resonance and ultrasound imaging system used in combination with a therapy system, in accordance with aspects of the present disclosure;

FIG. 5 depicts a process flow for determining a three-dimensional (3D) magnetic resonance (MR) volume specific to a respiration phase of a patient, in accordance with aspects of the present disclosure;

FIG. 6 depicts displacements over time of a tracked centroid corresponding to a vessel bifurcation, in accordance with aspects of the present disclosure;

FIG. 7 depicts output of a respiration phase cluster analysis, in accordance with aspects of the present disclosure; and

FIG. 8 depicts a process flow for using a 3D MR volume specific to a respiration phase of a patient in conjunction with real-time ultrasound beamspace data to gate a treatment performed on the patient, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Some generalized information is provided for both general context for aspects of the present disclosure and to facilitate understanding and explanation of certain of the technical concepts described herein.

Radiation therapy is a treatment option in which a stream of radiation (e.g., X-rays) is directed to a localized region of a patient identified for treatment, such as a tumor. Exposure to the directed stream of radiation may be intended to reduce the growth of or kill the targeted tissue. Because radiation therapy is typically employed to target internal regions within a patient, identifying the region to be targeted typically involves some form of non-invasive imaging.

For example, in conventional treatment processes, comparison of images and/or volumes captured before and during radiation therapy is typically done by aligning images using deformable registration methods. Such deformable registration techniques, however, are not feasible for real-time applications. Thus, insight gained into the efficacy of the treatment is not obtained in real-time, and may not be useful in guiding the process

Image-guided radiation therapy (IGRT) is the use of imaging during radiation therapy to improve the precision and accuracy of treatment delivery. As noted herein, tumors can shift inside the body, because of breathing and other movement. IGRT may allow doctors to locate and track tumors at the time of treatment and deliver more precise radiation treatment. This technology also allows radiation oncologists to make technical adjustments when a tumor moves outside of the planned treatment range. As a result, the radiation treatment is targeted to the tumor as much as possible, helping to limit radiation exposure to healthy tissue and reduce common radiation therapy side-effects.

The techniques disclosed herein may be used with IGRT and may utilize an ultrasound probe (e.g., a magnetic resonance (MR)-compatible three-dimension (3D) ultrasound (U/S) probe to simultaneously image an anatomy of interest using MR imaging and ultrasound and to thereby provide subject-specific respiratory data specific to the individual undergoing treatment. However, in certain implementations, such as those where IGRT is not performed, either 3D or two-dimensional (2D) ultrasound data may instead be acquired and used by the tracking and clustering routines discussed herein for respiratory state analysis. As discussed herein, the presently disclosed techniques automatically extract end-inspiration/expiration respiratory phases for precise dose-delivery in IGRT. By way of example, the techniques discussed herein may be used to track the respiratory phase of the patient using 4D ultrasound (longitudinal three-dimensional (3D) volumes over time (e.g., at ˜4 frames per second (FPS)) such that time is the fourth dimension) before and during radiation therapy. By way of example, and as discussed herein, respiratory phase tracking may be performed in one implementation by tracking one or more vessel bifurcations within an organ of interest. For instance, the subject-specific respiratory data may be comprised of the centroids of the tracked vessel bifurcation, which provides the displacements due to respiratory motion. As tumors are typically highly vascularized, tracking a vessel bifurcation within or around a tumor may be considered be analogous to tracking the tumor as the motions correlate well. With this motion information, the radiation dose is delivered at the end-inspiration or expiration phase of respiration, which may be considered the most reproducible phase in un-guided free breathing.

In certain embodiments, this result may be achieved in two steps. In a first step a deep learning (DL)-based process will automatically track vessel bifurcations (fiducials) within a region of interest (e.g., the liver) whose movements may be related to respiration and infer subject-specific respiratory data from the fiducial displacements due to respiration. The DL based respiration tracking methodology used to identify vessel bifurcations in ultrasound data may be agnostic to anatomy and may be extendible to applications other than IGRT. In a second step, a graph-based clustering technique may be employed on this respiratory data to automatically label the expiration phases. The labels may be further used to train a machine learning model with corresponding tracked displacements before therapy to automatically infer the expiration phases during therapy. The methodology of tracking+clustering+training/prediction the respiratory data into respiratory phases can be adopted for IGRT of cancers in any organ of the abdomen and lung. In one implementation, the analysis may be directly performed on ultrasound beam-space data to allow real-time tracking and prediction during treatment, as opposed to performing the analysis on scan-converted 4D ultrasound data.

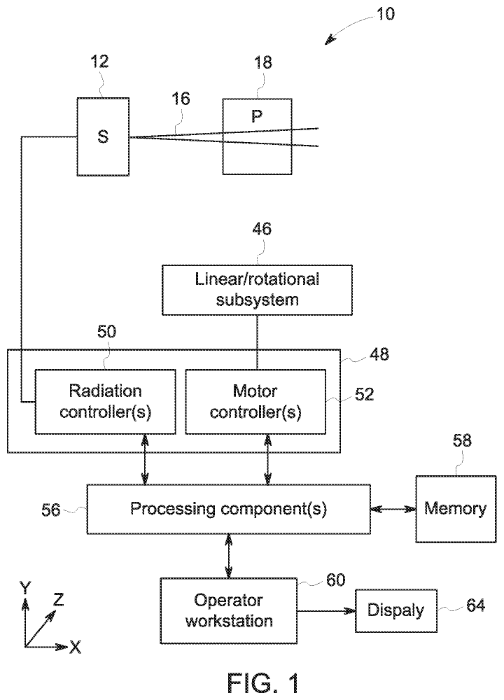

With the preceding in mind, an example of an IGRT system 10 suitable for use with the present techniques as discussed herein is provided in FIG. 1. In the embodiment illustrated in FIG. 1, IGRT system 10 includes a source of radiation 12 (e.g., X-rays or other types of radiation suitable for radiation therapy). The radiation source 12 may be an X-ray tube or any other source of radiation suitable for radiation therapy. The radiation 16 generated by the source 12 pass into a region in which a patient 18, is positioned during a treatment procedure. In the depicted example, the radiation 16 is collimated or otherwise shaped or limited to a suitable beam geometry which passes through the treatment volume.

In accordance with present embodiments, the radiation source 12 may be moved relative to the patient 18 along one or more axes during a treatment procedure during which radiation is directed toward a treatment region (e.g., a tumor). In one embodiment, the translation and/or rotation of the radiation source 12 may be determined or coordinated in accordance with a specified protocol and/or based upon an initial guidance trajectory based upon prior imaging of the patient 18.

The movement of the radiation source 12 may be initiated and/or controlled by one or more linear/rotational subsystems 46. The linear/rotational subsystems 46 may include support structures, motors, gears, bearings, and the like, that enable the rotational and/or translational movement of the radiation source 12. In one embodiment, the linear/rotational subsystems 46 may include a structural apparatus (e.g., a C-arm or other structural apparatus allowing rotational movement about at least one axis) supporting the radiation source 12.

A system controller 48 may govern the linear/rotational subsystems 46 that initiate and/or control the movement of the radiation source 12. In practice, the system controller 48 may incorporate one or more processing devices that include or communicate with tangible, non-transitory, machine readable media collectively storing instructions executable by the one or more processors to perform the operations described herein. The system controller 48 may also include features that control the timing of the activation of the radiation source 12, for example, to gate the emission of radiation based upon respiratory motion of the patient 18 (i.e., respiration gating). In general, the system controller 48 may be considered to command operation of the IGRT system 10 to execute treatment protocols. It should be noted that, to facilitate discussion, reference is made below to the system controller 48 as being the unit that controls source activation, movements, respiratory gating, and so forth, using the radiation source 12. However, embodiments where the system controller 48 acts in conjunction with other control devices (e.g., other control circuitry local or remote to the IGRT system 10) are also encompassed by the present disclosure.

In the present context, the system controller 48 includes signal processing circuitry and various other circuitry that enables the system controller 48 to control the operation of the radiation source 12 and the linear/rotational subsystems 46, such as to perform respiratory gating and/or radiation therapy as described herein. In the illustrated embodiment, the circuitry may include a source controller 50 configured to operate the radiation source 12. Circuitry of the system controller 48 may also include one or more motor controllers 52. The motor controllers 52 may control the activation of various components that are responsible for moving the radiation source 12. In other words, the motor controllers 52 may implement a particular treatment trajectory or motion for the radiation source 12.

It should be noted that the tangible, non-transitory, machine-readable media and the processors that are configured to perform the instructions stored on this media that are present in the IGRT system 10 may be shared between the various components of the system controller 48 or other components of the IGRT system 10. For instance, as illustrated, the radiation controller 50 and the motor controller 52 may share one or more processing components 56 that are each specifically configured to cooperate with one or more memory devices 58 storing instructions that, when executed by the processing components 56, perform IGRT techniques as discussed herein.

The system controller 48 and the various circuitry that it includes, as well as the processing and memory components 56, 58, may be accessed or otherwise controlled by an operator via an operator workstation 60. The operator workstation 60 may include any application-specific or general-purpose computer that may include one or more programs (for example one or more IGRT programs) capable of enabling operator input for the techniques described herein. The operator workstation 60 may include various input devices such as a mouse, a keyboard, a trackball, or any other similar feature that enables the operator to interact with the computer. The operator workstation 60 may enable the operator to control various imaging parameters, for example, by adjusting certain instructions stored on the memory devices 58. The operator workstation 60 may be communicatively coupled to a display 64 that enables the operator to view various parameters in real time, to view images produced by the image guidance functionality discussed herein, and the like.

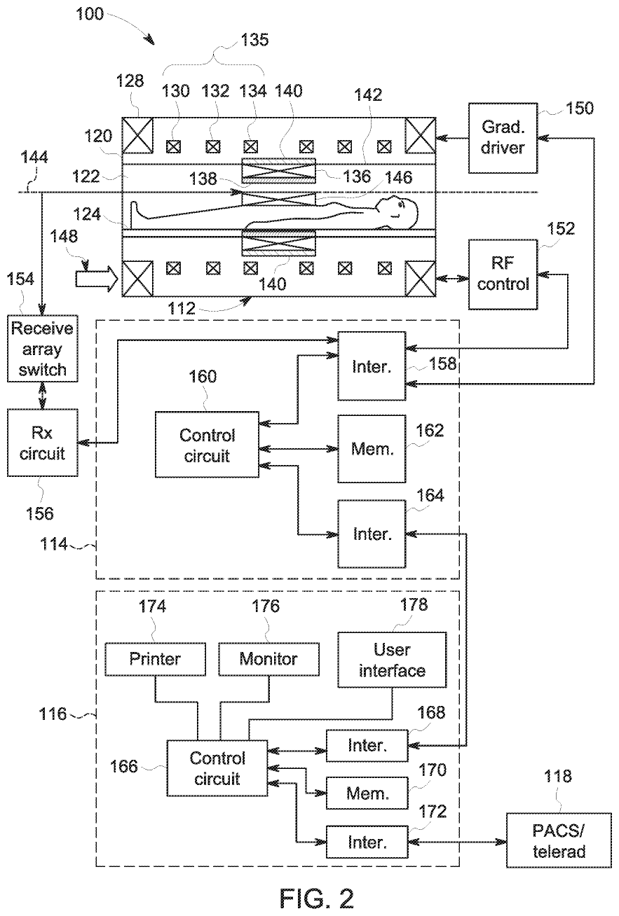

Various aspects of the present approaches may be further appreciated with respect to FIGS. 2-4. As discussed herein, an IGRT process is disclosed that includes at certain stages the simultaneous or near-simultaneous (e.g., temporally consecutive) acquisition of MR and ultrasound images in a pre-treatment phase. The pre-treatment ultrasound image data is integrated with information derived from the pre-treatment MR imaging, which provides useful tissue contrast. The present techniques, thus, include simultaneous or near-simultaneous pre-treatment MR and ultrasound imaging, which is then leveraged for guidance in an IGRT context.

With the preceding in mind, material related to imaging techniques and terms is provided below so as to impart some familiarity with such imaging systems and to provide useful real-world context for other aspects of the disclosure. With respect to magnetic resonance imaging (MM) systems, and turning to FIG. 2 where one such system is schematically illustrated for reference, interactions between a primary magnetic field, time varying magnetic gradient fields, and a radiofrequency (RF) field with gyromagnetic material(s) within a subject of interest (e.g., a patient) are used to generate images or volumetric representations of structural and/or functional relationships within the patient. Gyromagnetic materials, such as hydrogen nuclei in water molecules, have characteristic behaviors in response to externally applied electromagnetic fields (e.g., constant or time varying electric fields, magnetic fields, or a combination thereof) that may be leveraged in this manner. For example, the precession of spins of these nuclei can be influenced by manipulation of the fields to produce RF signals that can be detected, processed, and used to reconstruct a useful image.

With this in mind, and referring to FIG. 2, a magnetic resonance imaging system 100 is illustrated schematically as including a scanner 112, scanner control circuitry 114, and system control circuitry 116. The imaging system 100 additionally includes remote access and storage systems 118 and/or devices such as picture archiving and communication systems (PACS), or other devices such as teleradiology equipment so that data acquired by the imaging system 100 may be accessed on- or off-site. While the imaging system 100 may include any suitable scanner or detector, in the illustrated embodiment, the imaging system 100 includes a full body scanner 112 having a housing 120 through which an opening (e.g., an annular opening) is formed to accommodate a patient bore 122. The patient bore 122 may be made of any suitable material such as a non-metallic and/or non-magnetic material and generally includes components of the scanner 112 proximate to a subject. A table 124 is moveable into the patient bore 122 to permit a patient 18 to be positioned therein for imaging selected anatomy within the patient 18. As described herein, the patient bore 122 may include one or more bore tubes to support various components of the scanner 112 and/or the patient 18. In some embodiments, the patient bore 122 may support the table 124 and/or articulation components (e.g., a motor, pulley, and/or slides).

The scanner 112 may include a series of associated superconducting magnetic coils for producing controlled electromagnetic fields for exciting the gyromagnetic material within the anatomy of the subject being imaged. Specifically, a primary magnet coil 128 is provided for generating a primary magnetic field, which is generally aligned with an axis 144 of the patient bore 122. A series of gradient coils 130, 132, and 134 (collectively 135) permit controlled magnetic gradient fields to be generated for positional encoding of certain of the gyromagnetic nuclei within the patient 18 during examination sequences. An RF coil 136 is configured to generate radio frequency pulses for exciting the certain gyromagnetic nuclei within the patient 18. The RF coil 136 may be implemented on a coil support tube 138 defining at least a portion of the patient bore 122. Further, an RF shield 140 may be implemented on a shield support tube 142 also defining at least a portion of the patient bore 122 to reduce electromagnetic interference within the imaging system 100, as well as devices separate from the imaging system 100. In addition to the coils that may be local to the scanner 112, the imaging system 100 may also include a set of receiving coils 146 (e.g., an array of coils) configured for placement proximal (e.g., against) to the patient 18. As an example, the receiving coils 146 can include cervical/thoracic/lumbar (CTL) coils, head coils, single-sided spine coils, and so forth. Generally, the receiving coils 146 are placed close to or on top of the patient 18 so as to receive the weak RF signals (e.g., weak relative to the transmitted pulses generated by the scanner coils) that are generated by certain of the gyromagnetic nuclei within the patient 18 as they return to their relaxed state. In some embodiments, the RF coils 136 may both transmit and receive RF signals accomplishing the role of the receiving coils 146.

The various coils of the imaging system 100 are controlled by external circuitry to generate the desired field and pulses, and to read emissions from the gyromagnetic material in a controlled manner. In the illustrated embodiment, a main power supply 148 provides power to the primary magnetic coil 128 to generate the primary magnetic field. A driver circuit 150 may include amplification and control circuitry for supplying current to the gradient coils as defined by digitized pulse sequences output by the scanner control circuitry 114.

An RF control circuit 152 is provided for regulating operation of the RF coil 136. The RF control circuit 152 includes a switching device for alternating between the active and inactive modes of operation, wherein the RF coil 136 transmits and does not transmit signals, respectively. The RF control circuit 152 may also include amplification circuitry to generate the RF pulses. Similarly, the receiving coils 146, or RF coils 136 if no separate receiving coils 146 are implemented, are connected to a switch 154, which is capable of switching the receiving coils 146 between receiving and non-receiving modes. Thus, the receiving coils 146 may resonate with the RF signals produced by relaxing gyromagnetic nuclei from within the patient 18 while in the receiving mode, and avoid resonating with RF signals while in the non-receiving mode. Additionally, a receiving circuit 156 may receive the data detected by the receiving coils 146 and may include one or more multiplexing and/or amplification circuits.

It should be noted that while the scanner 112 and the control/amplification circuitry described above are illustrated as being connected by single lines, one or more cables or connectors may be used depending on implementation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner 112 and the scanner control circuitry 114 and/or system control circuitry 116.

As illustrated, the scanner control circuitry 114 includes an interface circuit 158, which outputs signals for driving the gradient coils 135 and the RF coil 136 and for receiving the data representative of the magnetic resonance signals produced in examination sequences. The interface circuit 158 may be connected to a control and analysis circuit 160. The control and analysis circuit 160 executes the commands to the driver circuit 150 and RF control circuit 152 based on defined protocols selected via system control circuitry 116.

The control and analysis circuit 160 may also serve to receive the magnetic resonance signals and perform subsequent processing before transmitting the data to system control circuitry 116. Scanner control circuitry 114 may also include one or more memory circuits 162, which store configuration parameters, pulse sequence descriptions, examination results, and so forth, during operation.

A second interface circuit 164 may connect the control and analysis circuit 160 to a system control circuit 166 for exchanging data between scanner control circuitry 114 and system control circuitry 116. The system control circuitry 116 may include a third interface circuit 168, which receives data from the scanner control circuitry 114 and transmits data and commands back to the scanner control circuitry 114. As with the control and analysis circuit 160, the system control circuit 166 may include a computer processing unit (CPU) in a multi-purpose or application specific computer or workstation. System control circuit 166 may include or be connected to a second memory circuit 170 to store programming code for operation of the imaging system 100 and to store the processed image data for later reconstruction, display and transmission. The programming code may execute one or more algorithms that, when executed by a processor, are configured to perform reconstruction of acquired data or other operations involving the acquired data.

An additional input output (I/O) interface 172 may be provided for exchanging image data, configuration parameters, and so forth with external system components such as remote access and storage systems 118. Finally, the system control circuit 166 may be communicatively coupled to various peripheral devices for facilitating an operator interface and for producing hard copies of the reconstructed images. In the illustrated embodiment, these peripherals include a printer 174, a monitor 176, and a user interface 178 including, for example, devices such as a keyboard, a mouse, a touchscreen (e.g., integrated with the monitor 176), and so forth.

In operation, a user (e.g., a radiologist) may configure and/or oversee control of the imaging system 100. Additionally, the user may assist in positioning the subject (e.g., a patient 18) within the patient bore 122. In some embodiments, the patient bore 122 may surround an entire subject or just a portion thereof (e.g., a patient's head, thorax, and/or extremity) while an imaging session is performed.

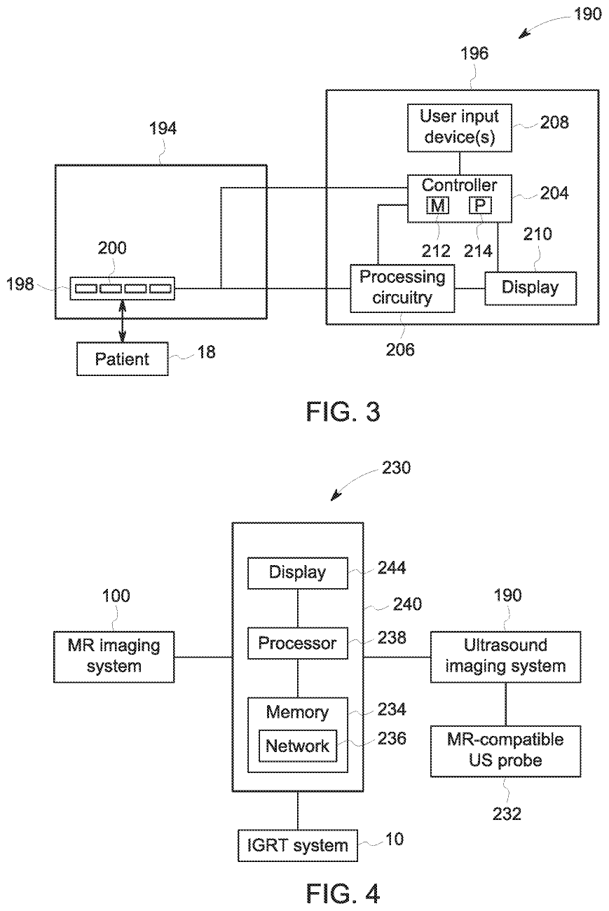

In addition to a Mill imaging system, certain examples discussed herein also utilize ultrasound data acquisition, such as to generate ultrasound images of the same anatomy of interest scanned using an Mill system 100. With this in mind, and to provide familiarity with aspects of such an ultrasound imaging system, FIG. 3 illustrates a block diagram of an embodiment of an ultrasound imaging system 190 capable of acquiring ultrasound data of a patient undergoing IGRT, such as prior to or during the procedure. In the illustrated embodiment, the ultrasound system 190 is a digital acquisition and beam former system, but in other embodiments, the ultrasound system 190 may be any suitable type of ultrasound system, not limited to the illustrated type. The ultrasound system 190 may include the ultrasound probe 194 and a workstation 196 (e.g., monitor, console, user interface) which may control operation of the ultrasound probe 194 and may process image data acquired by the ultrasound probe 194. The ultrasound probe 194 may be coupled to the workstation 196 by any suitable technique for communicating image data and control signals between the ultrasound probe 194 and the workstation 196 such as a wireless, optical, coaxial, or other suitable connection.

The ultrasound probe 194 contacts the patient 18 during an ultrasound examination. The ultrasound probe 194 may include a patient facing or contacting surface that includes a transducer array 198 having a plurality of transducer elements 200 capable of operating in a switched manner between transmit and receive modes. Each individual transducer element 200 may be capable of converting electrical energy into mechanical energy for transmission and mechanical energy into electrical energy for receiving. It should be noted that the transducer array 198 may be configured as a two-way transducer capable of transmitting ultrasound waves into and receiving such energy from a subject or patient 18 during operation when the ultrasound probe 194 is placed in contact with the patient 18. More specifically, the transducer elements 200 may convert electrical energy from the ultrasound probe 194 into ultrasound waves (e.g., ultrasound energy, acoustic waves) and transmit the ultrasound waves into the patient 18. The ultrasound waves may be reflected back toward the transducer array 198, such as from tissue of the patient 18, and the transducer elements 200 may convert the ultrasound energy received from the patient 18 (reflected signals or echoes) into electrical signals for processing by the ultrasound probe 194 and the workstation 196 to provide data that may be analyzed. The number of transducer elements 200 in the transducer array 198 and the frequencies at which the transducer elements 200 operate may vary depending on the application. In certain embodiments, the probe 194 may include additional elements not shown in FIG. 3, such as additional electronics, data acquisition, processing controls, and so forth.

As previously discussed, the ultrasound probe 194 is communicatively coupled to the workstation 196 of the ultrasound imaging system 190 to facilitate image collection and processing. As will be appreciated, the workstation 196 may include a number of components or features to control operation of the ultrasound probe 194, facilitate placement and/or guidance of the ultrasound probe 194, and facilitate production and/or interpretation of ultrasound data (including reconstructed ultrasound images). For instance, as illustrated, the workstation 196 may include a controller 204, processing circuitry 206, one or more user input devices 208, and a display 210. In certain embodiments, the workstation 196 may include additional elements not shown in FIG. 3, such as additional data acquisition and processing controls, additional image display panels, multiple user interfaces, and so forth.

The controller 204 may include a memory 212 and a processor 214. In some embodiments, the memory 212 may include one or more tangible, non-transitory, computer-readable media that store instructions executable by the processor 214 and/or data to be processed by the processor 214. For example, the memory 212 may include random access memory (RAM), read only memory (ROM), rewritable non-volatile memory such as flash memory, hard drives, optical discs, and/or the like. Additionally, the processor 214 may include one or more general purpose microprocessors, one or more application specific processors (ASICs), one or more field programmable logic arrays (FPGAs), or any combination thereof. The controller 204 may control transmission of the ultrasound waves into the patient 18 via the transducer array 198.

The processing circuitry 206 may include receiving and conversion circuitry. The processing circuitry 206 may receive the electrical signal data from the transducer array 198 of the ultrasound probe 194 representing reflected ultrasound energy returned from tissue interfaces within the patient 18. The processing circuitry 206 may process the data from the transducer array 198, such as correcting for noise artifacts, or the like. The processing circuitry 206 may then convert the signal data into an ultrasound image for presentation via the display 210 or to be used as an input to a respiration phase extraction process as discussed herein. Alternatively, in some embodiments, no image may be generated and the raw or unprocessed image data may be used as an input to a respiration phase extraction process as discussed herein. The controller 204 may cause display of the ultrasound image or images (or a construct or model generated based on such images or raw image data) produced by the processing circuitry 206 from the signal data received from the transducer array 198 of the ultrasound probe 194.

In operation, the controller 204 may receive a signal indicative of a target anatomy of the patient 18 and/or a target scan plane of the target anatomy via the one or more user input devices 208 of the workstation 196. The one or more user input devices 208 may include a keyboard, a touchscreen, a mouse, buttons, switches, or other devices suitable to allow the operator to input the target anatomy and/or the desired scan plane of the target anatomy. Based on the target anatomy and/or the target scan plane of the target anatomy, the controller 204 may output a signal to the transducer array 198 of the ultrasound probe 194 indicative of an instruction to convert the electrical energy from the ultrasound probe 194 into ultrasound waves and transmit the ultrasound waves into the patient 18 and to detect the ultrasound energy that is reflected back from the tissue interfaces within the patient 18.

With the preceding comments in mind, FIG. 4 illustrates a schematic diagram of an embodiment of a combined MR and ultrasound imaging system 230 that may be used for providing image-based guidance to an IGRT system 10, as described herein. The combined MR and ultrasound imaging system 230 includes a magnetic resonance (MR) imaging system 100 and an ultrasound imaging system 190. The ultrasound imaging system 190 may be communicatively coupled to a MR-compatible ultrasound probe 232. The MR-compatible ultrasound probe 232 may be an ultrasound probe configured for use in combination with the MR imaging system 100. As such, the MR-compatible ultrasound probe (as described in U.S. patent application Ser. No. 15/897,964, entitled “Magnetic Resonance Compatible Ultrasound Probe”, filed Feb. 15, 2018, which may be incorporated by reference in its entirety) may contain low or no ferromagnetic material (e.g., iron, nickel, cobalt) content.

In order to facilitate a simpler workflow, the ultrasound probe 232 may be capable of two-dimensional (2D) or three-dimensional (3D) volume acquisition with high temporal resolution, allowing an ultrasound image volume to be acquired at each of a sequence of time points. Moreover, besides being MR-compatible, the 3D ultrasound probe 232 may be electronically steerable and hands-free. This allows the ultrasound image field-of-view to be electronically manipulated, obviating the need for robotic or mechanical ultrasound probe holders to change the imaging field-of-view. In this manner, simultaneous MR and ultrasound images can be acquired. Moreover, during the treatment procedure (e.g., IGRT), the same ultrasound probe can be used and positioned in approximately the same manner as during the pre-treatment MR+ultrasound procedure without difficulty. This provides a further simplification of the workflow as approximately the same imaging set up is used between the pre-treatment and treatment procedure as the same ultrasound probe is utilized, and in the same manner. The data from the MR and ultrasound systems may be streamed to and stored in a memory system 234 which contains a trained network model 236, as discussed in greater detail herein, and which may be connected to other data storage or processing systems.

While the preceding describes relevant or utilized aspects of a combined imaging system as may be used prior to a treatment, other aspects of the system may be relevant or used during the procedure. By way of example, during the procedure an IGRT system 10 may be present and may leverage information gathered using the combined imaging system 230. The IGRT system 10, as discussed in greater detail below, may be guided by images obtained via the MR imaging system 100 in combination with images obtained via the ultrasound imaging system 190.

It should be noted that the system and process described herein entails two stages in the IGRT procedure or interventional procedure, a pre-treatment stage (e.g., patient-specific planning stage) where simultaneous MR and ultrasound imaging occurs, and a treatment stage or procedure (e.g., an IGRT phase) where ultrasound imaging occurs. With this in mind, the combined MR and ultrasound imaging system 230 may further include a system controller block 240 communicatively coupled to the other elements of the combined MR and ultrasound imaging system 230, including the MR imaging system 100, the ultrasound imaging system 190, and the IGRT system 10. The controller 240 may include a memory 234 and a processor 238. In some embodiments, the memory 234 may include one or more tangible, non-transitory, computer-readable media that store instructions executable by the processor 238 and/or data to be processed by the processor 238. For example, the memory 234 may include random access memory (RAM), read only memory (ROM), rewritable non-volatile memory such as flash memory, hard drives, optical discs, and/or the like. Additionally, the processor 238 may include one or more general purpose microprocessors, one or more graphic processing units (GPUs), one or more application specific processors (ASICs), one or more field programmable logic arrays (FPGAs), or any combination thereof. Further, the memory 234 may store instructions executable by the processor 238 to perform the methods described herein. Additionally, the memory 234 may store images obtained via the MR imaging system 100 and the ultrasound imaging system 190 and/or routines utilized by the processor 238 to help operate the IGRT system 10 based on image inputs from the MR imaging system 100 and the ultrasound imaging system 190, as discussed in greater detail below. The memory 234 may also store a neural network 236 that when trained tracks vessel bifurcations for use in subsequent processing steps for respiration phase determination. In certain embodiments, the system may be coupled to a remote database that includes the neural network 236 as opposed to directly incorporating the neural network 236. Further, the controller 240 may include a display 244 that may be used to display the images obtained by the MR imaging system 100 and/or the ultrasound imaging system 190.

With the preceding in mind, and as discussed herein, the present techniques employ a pre-treatment phase in which a patient-specific model may be trained to help identify respiration phases and a treatment phase in which the patient-specific model is used in conjunction with real-time ultrasound data to identify patient respiration phases in real-time and to use the respiration phase data to gate a treatment, such as a radiation therapy treatment. Thus, in one embodiment, 4D ultrasound may be used for tracking one or more organs and the tracking data may be used to extract expiration phases employed in respiration gating in image guided radiation therapy (IGRT). In certain such embodiments, ultrasound beam-space data (as opposed to reconstructed ultrasound images) may be used to perform organ tracking and expiration phase extraction, allowing real-time respiration tracking and gating. Further, 3D deep learning based vessel bifurcation tracking, as described herein, may be employed that further facilitates implementation of real-time capabilities required for IGRT. In particular, such data driven techniques are more generalizable and have much lower inference time compared to iterative methods conventionally employed, making the presently disclosed techniques more appropriate for real time application. However, it may be appreciated that the deep learning based tracking methodology described herein to identify vessel bifurcations in ultrasound data is agnostic to anatomy and may be extended to any application other than IGRT.

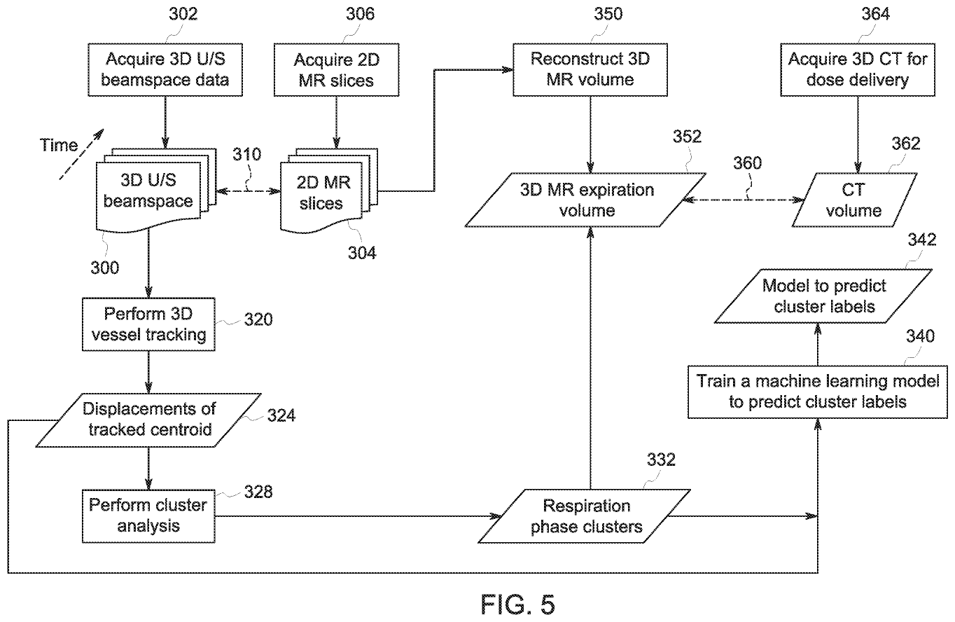

With the preceding in mind, and turning to FIGS. 5-8, process flows are provided that illustrate steps of one possible implementation of an IGRT process in accordance with the present disclosure. In this example, three-dimensional (3D) (or two-dimensional (2D) where appropriate) ultrasound (U/S) beamspace data 300 is acquired (step 302) over time simultaneous or contemporaneous with the acquisition (step 306) of two-dimensional (2D) MR slices. Based on the concurrence of the acquisition of the MR slices 304 and ultrasound beamspace data 300, data acquired at the same points in time may be matched or associated with one another, as indicated by dashed line 310.

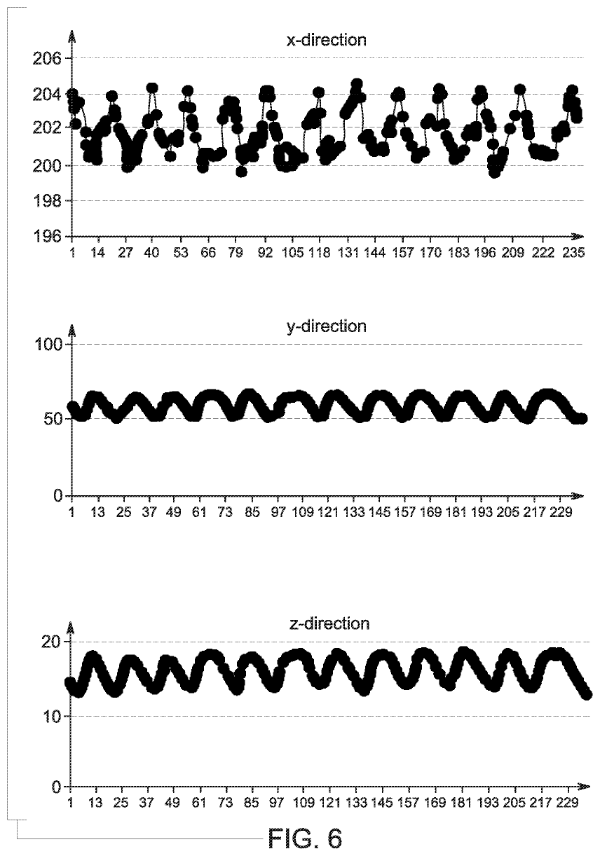

The 3D ultrasound beamspace data 300 may be provided as an input to a vessel tracking routine or process 320. In one implementation, the vessel tracking routine or process uses the 3D ultrasound beamspace data 300 to identify vessel bifurcations within a vascularized organ that moves in response to respiration, such as the liver. The vessel bifurcations may serve as fiducial markers that can be tracked over time and used by the vessel tracking routine to measure motion in the organ that is caused by or otherwise associated with respiration. By way of example, the 3D ultrasound beamspace data 300 may be provided as an input to a deep learning model (e.g., a neural network) that is configured to perform tracking of vessel bifurcations that may be used or processed to extract respiration phases, i.e., an expiration phase, over time using a clustering method. For instance, an output of the 3D vessel tracking step 320 may be a set of displacements of a tracked centroid 324 (which may correspond to a vessel bifurcation) from which respiration phases may be determined. As the imaging system data is for a particular subject of interest preparing to undergo a therapy procedure, the tracking outputs derived from the deep learning model at step 320 are specific to the current subject. That is, the subject-specific respiratory data, as discussed herein, may be comprised of the centroids of the tracked vessel bifurcation that provides the displacement data associated with respiratory motion for a given patient. An example of such a set of tracked centroid displacements over time is illustrated in FIG. 6.

In the depicted implementation, the set of tracked centroid displacements 324 is subjected to analysis to identify respiratory phases. By way of example, the tracked centroid displacements 324 may be subjected to cluster analysis (step 328), such as graph-based cluster analysis. Examples of graph-based clustering methods include, but are not limited to: hierarchical clustering, agglomerative clustering, spectral clustering, and so forth. Such graph-based clustering techniques may be used to group the tracked respiratory data (e.g., the set of tracked centroid displacements 324) into separate clusters representative of distinct respiratory phases.

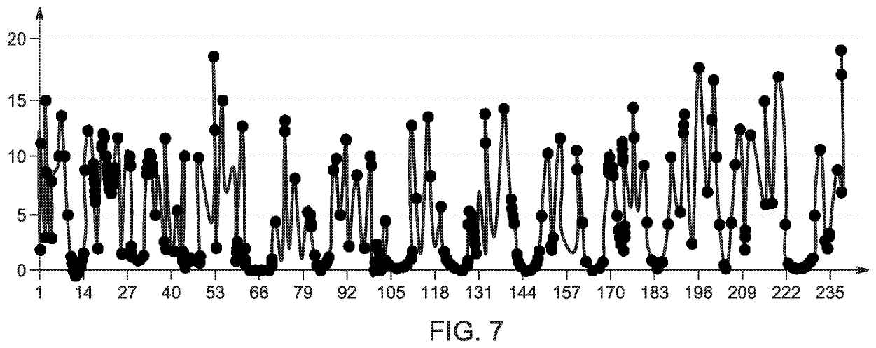

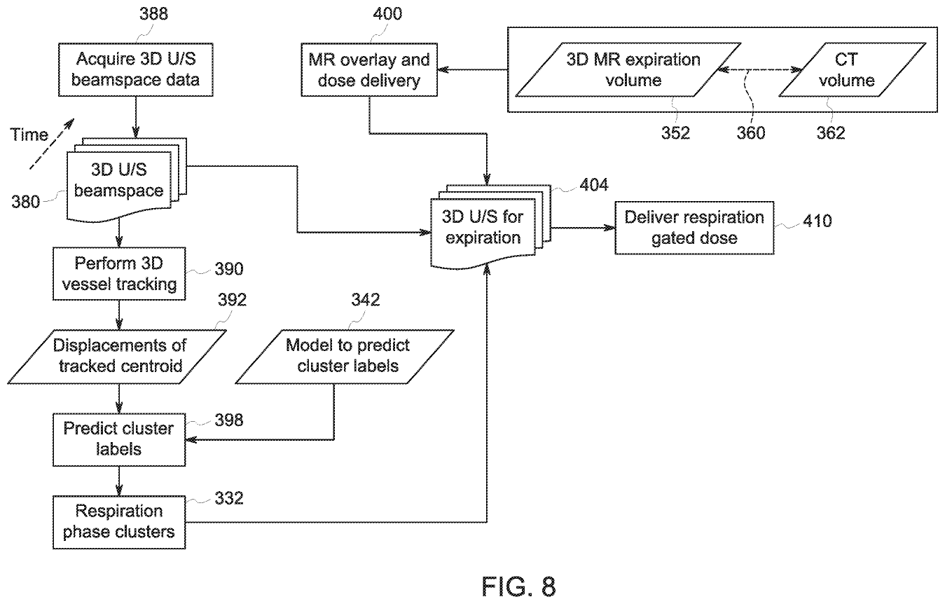

In one embodiment, the graph-based clustering for respiratory phase extraction uses vector cosine distances for measuring fiducial displacements as determined from beam-space ultrasound data, which is in non-rectilinear co-ordinate space. The vector cosine distances of the tracked centroids over the respiratory data are used to form a similarity graph, which may be used in assigning or clustering respiratory phases. An example of the output of a suitable graph-based cluster analysis may be one or more respiration phase clusters 332, an example of which is shown in FIG. 7. In the depicted example, the tracked centroid displacements 324 and respiration phase clusters 332 may be employed to train (step 340) a machine learning model 342, such as using a supervised training approach) to predict cluster labels using the displacements 324.

As expiration is the most reproducible phase in free-breathing, the cluster label with maximum number of data points is considered to be the expiration phase. Thus, in the example shown in FIG. 7, the cluster corresponding to zero (0) on the y-axis corresponds to an expiration state. As described herein, expiration phases will be reproducible before and during the therapy and use of the expiration phases with thereby eliminate the need to align before- and during-therapy ultrasound images to align the target region.

Expiration phases and other clusters labeled for ultrasound data before therapy can then be used to train a supervised machine learning model (e.g., support vector machines, k-nearest neighbor, random forest, logistic regression, etc.), to automatically label clusters from ultrasound data during therapy. The prediction routines are fast to execute and may be implemented in real-time. The prediction routines may further eliminate the need to re-cluster tracked vessel bifurcation(s) to determine respiratory states from ultrasound volumes for the same subject during therapy.

Turning back to FIG. 5, the acquired MR image data 304 may be reconstructed (step 350) to generate a 3D volume that may be merged or otherwise associated with the respiration phase clusters 332 (e.g., expiration phases) to generate, in this example, a 3D MR expiration volume 352. The 3D MR expiration volume 352 may, therefore, relate the appearance of the MR volume for a given patient with how it appears at a given respiratory phase, here expiration. As may be appreciated, this may be accomplished or otherwise facilitated using the known timing relationship 310 that relates the 2D MR slice data 304 used to reconstruct the volume 352 with the 3D ultrasound beamspace data from which respiratory phases 332 are determined.

In a further aspect, the 3D MR expiration volume 352 (or, in a more general example, a 3D MR respiration phase mapped volume) is registered (as denoted by dashed line 360) with a separately acquired CT volume 362. The CT volume 362 may be acquired (step 364) to facilitate radiation dose delivery during a therapy procedure. For example, the CT volume 362 may depict anatomic structure at a level of detail suitable for dose delivery so that tissue to be treated is targeted while other tissue is minimally impacted.

While FIG. 5 depicts steps used in a pre-therapy or pre-treatment context, FIG. 8, depicts a process flow illustrating steps that may be performed during therapy or treatment of a patient (e.g., radiation therapy). In this example, certain data or constructs from the pre-therapy process flow are carried forward. In particular, the registered (as denoted by dashed line 360) CT volume 362 and 3D MR expiration volume 352 are available for an MR overlay and dose delivery process 400.

In particular, in the depicted example 3D ultrasound beamspace data 380 is acquired (step 388) over time during a therapy procedure. In practice the acquisition may occur using the same probe and placement as was used during the pre-therapy procedure (i.e., the pre-therapy procedure may transition to the therapy procedure without removing or moving the ultrasound probe). The 3D ultrasound beamspace data 380 acquired in real-time during therapy may be provided as an input to a routine or deep learning model for 3D vessel tracking (step 390), as described herein, to derive displacements of tracked centroids 392 indicative of vessel bifurcations. As described above, the centroid data 392 may be subjected to the trained machine learning model 342 at step 398 to predict cluster labels to derive respiration clusters 396 (e.g., expiration phase data and timing). Thus, in real-time during therapy the 3D ultrasound beamspace data may be used to determine expiration phases of the subject.

The real-time expiration phase data (respiration phase clusters 332) may be used in conjunction with the previously determined 3D MR expiration volume 352, which is registered to the CT structural anatomy data 362, for targeting in real-time a region of interest with the therapy (e.g., radiation therapy). In particular, the pre-therapy 3D MR expiration volume 352 and registered CT volume 362 provide the geometric location and/or orientation of the target region during the respiratory phase of interest (e.g., expiration). Knowing this targeting information in conjunction with the real-time respiration phase (i.e., when the patient is in an expiration phase) from real-time ultrasound data, allows 3D ultrasound expiration data 404 to be used to deliver step (410) therapy (e.g., radiation dose) in a respiration gated manner and without guided breathing.

Technical effects of the invention include automatically determining respiratory phases (e.g., end-inspiration/expiration respiratory phases) in real time using ultrasound beamspace data. The respiratory phases may be used subsequently in a therapy or treatment (e.g., image-guided radiation-therapy (IGRT)) for precise dose-delivery. In certain implementations, three-dimensional, deep learning based vessel bifurcation tracking facilitates the real-time capabilities required for IGRT. In such implementations, the deep learning based tracking identifies vessel bifurcations in the ultrasound data and is agnostic to anatomy, allowing the methodology to be extended to applications other than IGRT.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method to generate a patient-specific respiration model, comprising the steps of:
acquiring magnetic resonance (MR) image data and ultrasound beamspace data of a patient over time;
in the ultrasound beamspace data, tracking one or more vascular vessel bifurcations over time;
based on a measure of the tracked vascular vessel bifurcations, determining one or more respiration phases of the patient over time;
generating a 3D MR volume from the MR image data; and
for a respective respiration phase of the one or more respiration phases, determining a 3D MR respiration phase volume for the patient from the 3D MR volume and specific to the respective respiration phase.

2. The method of claim 1, wherein the ultrasound beamspace data comprises two-dimensional (2D) ultrasound beamspace data or three-dimensional (3D) ultrasound beamspace data.

3. The method of claim 1, wherein the MR image data and the ultrasound beamspace data are acquired concurrently.

4. The method of claim 1, wherein the measure of the tracked vascular vessel bifurcations comprises displacements of one or more centroids corresponding to the one or more vascular vessel bifurcations.

5. The method of claim 1, wherein tracking the one or more vascular vessel bifurcations over time comprises:
providing the ultrasound beamspace data to a neural network trained to track vessel bifurcations over time and to output displacements of one or more centroids corresponding to the one or more vascular vessel bifurcations.

6. The method of claim 1, wherein determining the one or more respiration phases of the patient over time comprises:
performing a cluster analysis using the measure of the tracked vascular vessel bifurcations to identify one or more clusters, wherein each cluster corresponds to a respiration phase of the patient.

7. The method of claim 5, wherein the cluster analysis comprises a graph-based cluster analysis.

8. The method of claim 5, further comprising training a supervised machine learning model using displacement of the tracked vascular vessel bifurcations and corresponding cluster labels.

9. The method of claim 1, further comprising:
registering the three-dimensional MR respiration phase volume to a CT volume containing a target anatomic region for a treatment.

10. The method of claim 1, wherein the respective respiration phase comprises an expiration phase.

11. A method for respiration gating a patient treatment, comprising:
during a treatment procedure, acquiring ultrasound beamspace data of a patient over time;
in the ultrasound beamspace data, tracking one or more vascular vessel bifurcations over time;
based on a measure of the tracked vascular vessel bifurcations, predicting a series of activation respiration phases of the patient during the treatment;
determining one or both of a location or orientation of a target anatomic region during the activation respiration phases of the patient using a 3D MR respiration phase volume for the patient generated prior to the treatment procedure and specific to the activation respiration phase; and
applying the treatment to the target anatomic region during the series of activation respiration phases.

12. The method of claim 11, wherein the series of activation respiration phases of the patient during the treatment are predicted using a machine learning model trained using displacement of the tracked vascular vessel bifurcations and corresponding cluster labels.

13. The method of claim 11, wherein the ultrasound beamspace data comprises two-dimensional (2D) ultrasound beamspace data or three-dimensional (3D) ultrasound beamspace data.

14. The method of claim 11, wherein the 3D MR respiration phase volume for the patient is generated using concurrently acquired MR image data and ultrasound beamspace data of the patient over time prior to the treatment procedure.

15. The method of claim 11, wherein the measure of the tracked vascular vessel bifurcations comprises displacements of one or more centroids corresponding to the one or more vascular vessel bifurcations.

16. The method of claim 11, wherein tracking the one or more vascular vessel bifurcations over time comprises:
providing the ultrasound beamspace data to a neural network trained to track vessel bifurcations over time and to output displacements of one or more centroids corresponding to the one or more vascular vessel bifurcations.

17. The method of claim 11, wherein the 3D MR respiration phase volume for the patient is registered to a CT volume containing the target anatomic region for the treatment.

18. An image guided treatment system comprising:
a memory encoding processor-executable routines; and
a processing component configured to access the memory and execute the processor-executable routines, wherein the routines, when executed by the processing component, cause the processing component to perform actions comprising:
acquiring magnetic resonance (MR) image data and three-dimensional (3D) ultrasound beamspace data of a patient over time;
in the 3D ultrasound beamspace data, tracking one or more vascular vessel bifurcations over time;
based on a measure of the tracked vascular vessel bifurcations, determining one or more respiration phases of the patient over time;
generating a 3D MR volume from the MR image data;
for a respective respiration phase of the one or more respiration phases, determining a 3D MR respiration phase volume for the patient from the 3D MR volume and specific to the respective respiration phase; and
respiration gating application of a treatment to the patient using the 3D MR respiration phase volume and an indication of respiration phase of the patient determined using 3D ultrasound beamspace data acquired during the application of the treatment.

19. The image guided treatment system of claim 18, further comprising a radiation emitting component configured to emit radiation during the treatment.

20. The image guided treatment system of claim 18, wherein tracking the one or more vascular vessel bifurcations over time comprises:
providing the ultrasound beamspace data to a neural network trained to track vessel bifurcations over time and to output displacements of one or more centroids corresponding to the one or more vascular vessel bifurcations.

21. The image guided treatment system of claim 18, wherein determining the one or more respiration phases of the patient over time comprises:
performing a cluster analysis using the measure of the tracked vascular vessel bifurcations to identify one or more clusters, wherein each cluster corresponds to a respiration phase of the patient
